(19) United States
(12) Patent Application Publication
(10) Pub. No.: US 2005/0158745 A1
(43) Pub. Date: Jul. 21, 2005

(54) Methods and compositions for detection of nasopharyngeal carcinoma

(76) Inventor: Jack Zhihai Ye, Danville, CA (US)

Correspondence Address: PERKINS COIE LLP, POST OFFICE BOX 1208, SEATTLE, WA, 98111-1208, US

(21) Appl. No.: 10/996,176

(22) Filed: Nov. 23, 2004

Related U.S. Application Data
(60) Provisional application No. 60/481,701, filed on Nov. 25, 2003.

(51) Int. Cl. C12Q 1/68; G01N 33/574; C12M 1/34

(52) U.S. Cl. 435/6; 435/7.23; 435/287.2

(57) ABSTRACT

This invention involves systematic identification of nasopharyngeal carcinoma biomarker protein panels which effectively distinguish serum samples from patients and normal individuals. It uses a combination of protein chip technology in conjunction with surface-enhanced laser desorption/ionization time-of-flight mass spectrometry (SELDI-TOF-MS) procedures. The analysis of serum samples of nasopharyngeal carcinoma patients and normal ones revealed significant differences among separate protein and peptide species examined and indicated either the presence or absence of nasopharyngeal carcinoma by a said pattern of biomarkers.

### CROSS-REFERENCE TO RELATED APPLICATIONS

This application claims priority to U.S. Provisional Patent Application No. 60/481,701 filed Nov. 25, 2003, which is hereby incorporated by reference in its entirety including drawings as fully set forth herein.

### FIELD OF THE INVENTION

The present application relates to the field of proteomics and cancer diagnosis. In particular, the present application relates to biomarkers specific to nasopharyngeal carcinoma and methods of use thereof.

### BACKGROUND OF THE INVENTION

Proteomic approaches have been used at identifying differences in protein expression pattern. These potential protein alterations can be used as new cancer biomarkers for cancer diagnosis.

Initially, two-dimensional PAGE has been used to detect differentially expressed proteins for some time. Advances in 2D-PAGE technology coupled with software programs and tandem mass spectrometry microsequencing have improved this proteomic system. However, this approach resolves low-abundant proteins poorly, and often suffers reproducibility problems. In addition, this approach can not detect low molecular weight proteins and/or peptides. Therefore, the 2D-PAGE technology is not ready to be transformed into a clinical assay yet.

Recently, matrix-assisted laser desorption ionization time-of-flight (MALDI-TOF) has emerged as a more advanced technology to achieve high-throughput separation and analysis of multiple proteins in a sample. It is potentially faster and more comprehensive than 2D-PAGE for comparison of protein profiles between samples.

More recently, surface enhanced laser desorption ionization time-of-flight mass spectrometry (SELDI-TOF MS) has been described as modification of MALDI-TOF in which small amounts of proteins are captured, by absorption, partition, electrostatic interaction or affinity chromatography on a stationary-phase and immobilized in an array format on a protein chip coated with specific chemical matrices (hydrophobic, cationic, anionic, normal phase etc). The bound subjects are then ionized by matrix-assisted laser desorption/ionization and their mass-to-charge (M/Z) ratios measured by TOF MS (mass spectrometry). The result is simply a mass spectrum of the subjects that bound to and subsequently desorbed from the array. Consequently, mixtures of proteins and peptides can be resolved as fingerprint of the sample.

Due to the nature of carcinogenesis whereas early onset of cancers usually contributes variety of protein and/or peptide changes within patient's organs and tissues, particularly in patient's body fluids such as blood or serum, SELDI-TOF MS has been used successfully to identify biomarkers of ovarian, prostate, breast and pancreatic cancers.

U.S. Pat. No. 6,020,208 and No. 6,225,047 described a protein chip technology for multiple sample presentation, Surfaces Enhanced for Laser Desorption/Ionization (SELDI). The SELDI process pre-selects analyte molecules in a sample by allowing them to bind to the treated surface of a metal bar coated with a specific chemical that binds a subset of the proteins or peptides within a blood sample. The attached molecules are subsequently detached in a lasor-dependent manner.

Liotta et al, Lancet (2002) 359:572-577, demonstrated that SELDI-TOF masses spectrometry can be successfully used to the discovery of a particular proteomic pattern, which completely discriminates ovarian cancers from non-cancers. Such pattern for early ovarian cancer diagnosis consists of 5 peptides, with M/Z values of 534, 989, 2111, 2251 and 2465. The result yielded 100% sensitivity and 95% specificity. The positive predictive value was 94%, compared with 35% for CA125, currently used for ovarian cancer diagnosis, for the same set of samples.

More recently, the diagnosis of several other types of cancer have been reported using SELDI-TOF. Proteomic patterns for breast cancer was demonstrated by Du Bois et al, British Journal of Cancer (2002) 86:1440-1443, Petricoin et al, Disease Markers (2001) 17:301-307, Chan et al, Clinical Chemistry (2002) 48:1296-1304. Biomarker pattern for prostate cancer were reported separately by Wright et al, Cancer Research (2002) 62:3609-3614 and Petricoin, Liotta et al, Journal of National Cancer Institute (2002) 94:1576-1578. Biomarkers for hepatocellular carcinoma was shown by Poon, Johnson P J et al, Clinical Chemistry (2003) 49:752-760.

On the other hand, nasopharyngeal carcinoma (or NPC) is one the leading malignancy in both men and women in the Southeast Asia, but also seen throughout the world. Squamous cell carcinoma is the most common one seen in the nasopharynx, nose, and paranasal sinuses, and tend to remain asymptomatic until late in their course. Despite some diagnostic methods, such as CT or NMR image scanning and biopsy, have been used for its early detection, they are still relatively ineffective at identifying early and smaller potentially curable cancer lesions. So far, there is no cancer biomarker specific for nasopharyngeal carcinoma being found and used in clinics yet. Therefore, sensitive and specific biomarkers are badly needed to accomplish the early diagnosis of nasopharyngeal carcinoma and improve the prognosis of the patients.

### SUMMARY OF THE INVENTION

One aspect of the present invention is directed to at least one biomarker specific to nasopharyngeal carcinoma in a patient, wherein the biomarker comprises a polypeptide having a M/Z value of 28383+/−142, 23650+/−118, 16128+/−81, 15741+/−79, 15310+/−77, 13930+/−70, 11836+/−59, 8998+/−45, 5856+/−29, 5690+/−28, 5531+/−28, 5391+/−27, 4172+/−24, 3208+/−16, or 2833+/−14 on protein chip array of WCX2, or a combination thereof. In a preferred embodiment, the biomarker is at least one polypeptide having a M/Z value selected from the group consisting of 23650+/−118, 16128+/−81, 8998+/−45, 5391+/−27, and a combination thereof. In a more preferred embodiment, the biomarker is at least one polypeptide having a M/Z value selected from the group consisting of 23650, 16128, 8998, 5391, and a combination thereof. The discovery of biomarkers results from combining protein chip technology and SELDI-TOF-MS (surface enhanced laser desorption ionization time-of-flight mass spectrometry). The identified biomarkers act as benchmarks or indicators for early diagnosis of nasopharyngeal carcinoma, treatment, monitoring, and detection of cancer recurrence.

Another aspect of the present invention is directed to methods for identifying a biomarker or a panel of biomarkers for nasopharyngeal carcinoma comprising: a) collecting a first set of blood samples, particularly serum samples, from confirmed nasopharyngeal carcinoma patients; b) collecting a second set of serum samples from noncancerous patients or normal people; c) conducting SELDI-TOF-MS analysis for the first and second sets of serum samples; d) compare the data collected between the two serum sample sets; wherein differences in the profiles are indicative of the identification of biomarkers specific for nasopharyngeal carcinoma.

One embodiment of the present invention is directed to the establishment of a biomarker reference comprising the steps of comparing analyzed data of serum samples from malignant tumor and normal control group populations and developing a constituent panel to show differences in samples.

In another embodiment, a testing sample may be from any specimen that is in liquid form. A non-limiting example of a testing sample is a serum sample. In a preferred embodiment of the invention, a noncancerous sample may be a serum sample from a normal individual or a patient. In another preferred embodiment, a cancerous sample may be a serum sample mainly used and concerned in this invention or any other body fluid from a nasopharyngeal carcinoma patient.

Yet in another embodiment, the data from the SELDI-TOF-MS analysis can be compared through a training algorithm which is employed to compare the protein marker profiles between cancerous and noncancerous patients. One example of the training algorithm contains means for plotting and analyzing the protein patterns as clusters or groups that are similar or not similar. After training, the pattern of an unknown sample is diagnostically classified by its similarity to the diseased or normal clusters found in the training set.

Yet in another embodiment, a plurality of cancer biomarkers in the serum of a nasopharyngeal carcinoma patient were identified. The optimum discriminatory pattern for nasopharyngeal carcinoma was defined by the amplitudes or intensity at the key M/Z (mass-to-charge ration) values. As a result of these procedures, the nasopharyngeal carcinoma specific biomarkers were found as M/Z values of 28383+/−142, 23650+/−18, 16128+/−81, 15741+/−79, 15310+/−77, 13930+/−70, 11836+/−59, 8998+/−45, 5856+/−29, 5690+/−28, 5531+/−28, 5391+/−27, 4172+/−24, 3208+/−16, 2833+/−14 on protein chip array of WCX2. In a preferred embodiment, the biomarker is at least one polypeptide having a M/Z value selected from the group consisting of 23650+/−118, 16128+/−81, 8998+/−45, 5391+/−27, and a combination thereof. In a more preferred embodiment, the biomarker is at least one polypeptide having a M/Z value selected from the group consisting of 23650, 16128, 8998, 5391, and a combination thereof. These markers can be used as the direct basis for diagnosis, prognosis, or treatment monitoring of a patient with nasopharyngeal carcinoma.

Yet in another embodiment, the biomarker panel of pharyngeal carcinoma includes intensity value ranges for each biomarker. The method of using the intensity value of a protein or peptide to predict or diagnose pharyngeal carcinoma comprising the steps of using serum sample from an individual to provide a method of cancer diagnosis; comparing intensity value of protein biomarker with a reference protein intensity value; and determining the alteration of intensity value of said individual protein biomarker over said reference protein to diagnose said subject.

In a preferred embodiment, the diagnosis of nasopharyngeal carcinoma can be determined through the following three scenarios (shown in FIG. 6): 1) When the intensity ratio of protein or peptide of M/Z of 23650+/−118 is less or equal to 2.7, the patient can be diagnosed as having nasopharyngeal carcinoma. 2) When the intensity ratio of protein or peptide of M/Z of 23650+/−118 is higher than 2.8, and the intensity ratio of protein of M/Z of 16128+/−81 is higher than 8.1, and the intensity ratio of protein of M/Z of 8998+/−45 is higher than 7.1, then the patient can be diagnosed as having nasopharyngeal carcinoma. 3) When the intensity ratio of protein or peptide of M/Z of 23650+/−118 is higher than 2.8, and the intensity ratio of protein of M/Z of 16128+/−81 is higher than 8.1, and the intensity ratio of protein of M/Z of 8998+/−45 is higher than 7.1, and the intensity ratio of protein of M/Z of 5391+/−27 is less or equal to 6.6, then the patient can be diagnosed as having nasopharyngeal carcinoma.

Another aspect of the present invention is directed to the use of a combination of protein chip technology in conjunction with SELDI-TOF-MS detection procedures to maximize the diversity of biomarkers which are verifiable within a particular sample. The cohort of biomarkers verified within a sample is then viewed with reference to their ability to evidence at least one particular disease state; thereby enabling a diagnostician to gain the ability to characterize either the presence or absence of said at least one disease state relative to recognition of the presence and/or the absence of said biomarkers.

Another aspect of the present invention is directed to a method to identify specific biomarkers for nasopharyngeal carcinoma comprises the steps of a) semi-purifying potential biomarkers using micro spin columns; b) eluting proteins bound on the column and analyzing on a normal phase protein chip array; c) enzymatically digesting fractions containing potential biomarkers through in-gel tryptic digestion; d) resolving the resultant tryptic peptides from c) by ProteinChip Reader and analyzing by the a software; e) obtaining protein ID or protein/peptide sequence by searching NCBI database using ProFound search engine.

Another aspect of the present invention is directed to a method for identifying or diagnosing nasopharyngeal carcinoma in a subject comprising collecting a blood sample from a subject suspected of having nasopharyngeal carcinoma, conducting SELDI-TOF-MS analysis for the blood sample and a standard blood sample, compare the data collected between the two samples; wherein a difference between the blood sample and the standard sample in at least one biomarker specific for nasopharyngeal carcinoma is indicative of the propensity for the subject having nasopharyngeal carcinoma. In a preferred embodiment, the biomarker is a polypeptide having a M/Z value of 5391+/−27, 8998+/−45, 16128+/−81, or 23650+/−118 or a combination thereof. In another preferred embodiment, the difference is the intensity or intensity ratio as described herein.

Another aspect of the present invention is directed to a method for identifying or determining regression, progression or onset of nasopharyngeal carcinoma comprising the steps of collecting a blood sample from a subject having or suspected of having nasopharyngeal carcinoma, conducting SELDI-TOF-MS analysis for the blood sample and a standard blood sample, comparing the data collected between the two samples; wherein a difference between the blood sample and the standard sample in at least one biomarker specific for nasopharyngeal carcinoma is indicative of regression, progression or onset of nasopharyngeal carcinoma.

Another aspect of the present invention is directed to a method for evaluating the effect of a drug candidate for nasopharyngeal carcinoma comprising collecting a blood sample from a subject having nasopharyngeal carcinoma and being administered with the drug candidate, conducting SELDI-TOF-MS analysis for the blood sample and a standard blood sample, comparing the data collected between the two samples; wherein the reducing, sustaining or increasing of a difference between the blood sample and the standard sample in at least one biomarker specific for nasopharyngeal carcinoma is indicative of the effect of the drug candidate.

Another aspect of the present invention is directed to a post-operative method to monitor cancer prognosis and occurrence comprises: using a serum sample from said subject to develop a post-operative biomarker panel; comparing said post-operative biomarker panel with a pre-operative biomarker reference panel for said subject; and determining the absence or still presence of malignancy by monitoring at least one constituent of said biomarker panels.

Another aspect of the present invention is directed to antibodies raised against an identified polypeptide for a biomarker specific to nasopharyngeal carcinoma. In a preferred embodiment, the antibody is a polyclonal antibody, a monoclonal antibody, a humanied antibody, or a fraction thereof.

Another aspect of the present invention is directed to the use of identified, isolated biomarker as a vaccine against nasopharyngeal carcinoma. Another aspect of the present invention is directed to a diagnostic kit for determining the presence of said disease specific marker comprising an antibody against at least one biomarker specific to nasopharyngeal carcinoma.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a representative example of derived SELDI-TOF-MS spectrum which characterizes samples from nasopharyngeal carcinoma (NPC) and normal individuals (normal).

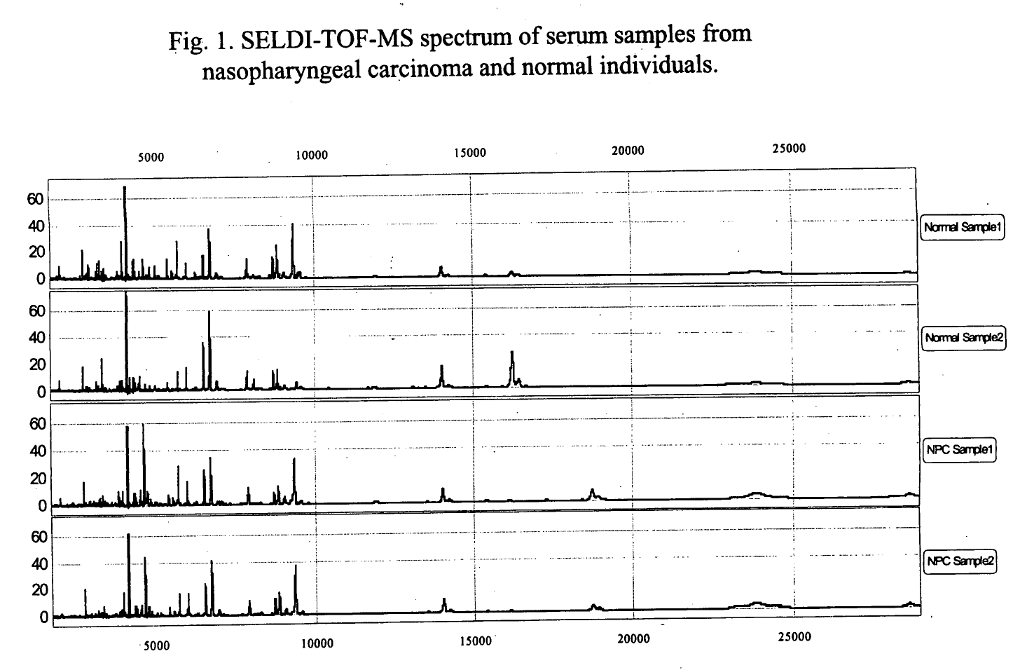

FIG. 2 is a representative SELDI-TOF-MS spectrum which characterizes a biomarker for nasopharyngeal carcinoma with the molecular weight (M/Z value) of 23650.

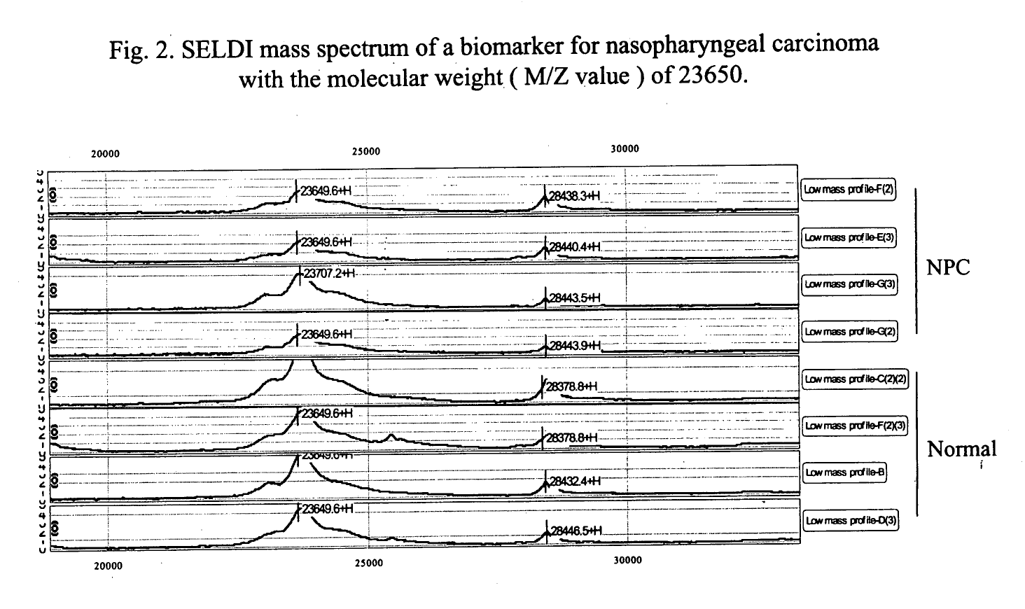

FIG. 3 is a representative SELDI-TOF-MS spectrum which characterizes a biomarker for nasopharyngeal carcinoma with the molecular weight (M/Z value) of 16128.

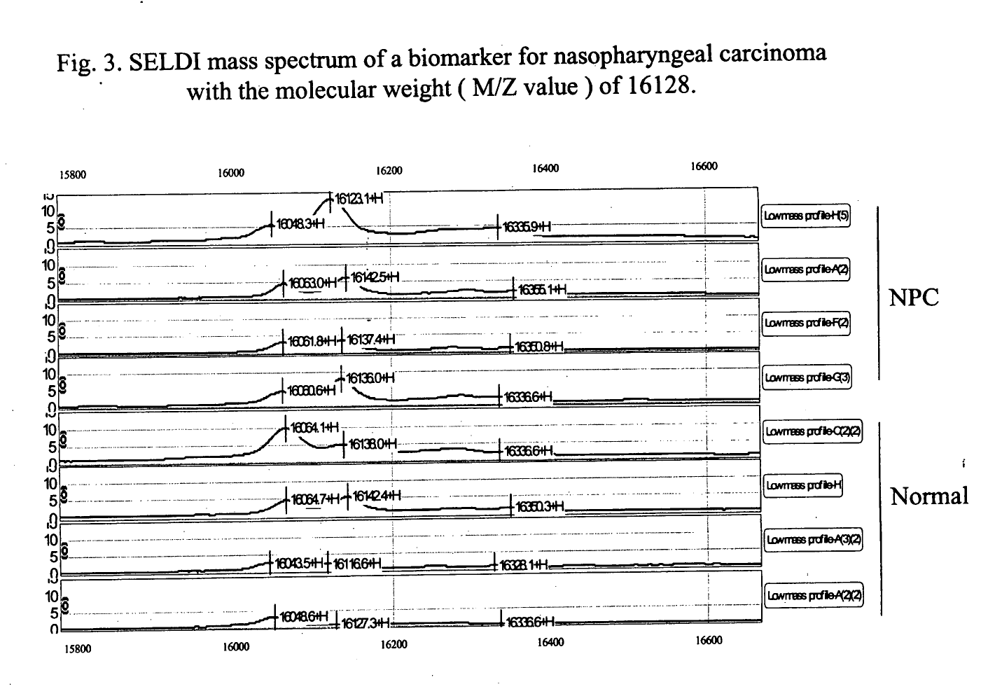

FIG. 4 is a representative SELDI-TOF-MS spectrum which characterizes a biomarker for nasopharyngeal carcinoma with the molecular weight (M/Z value) of 8998.

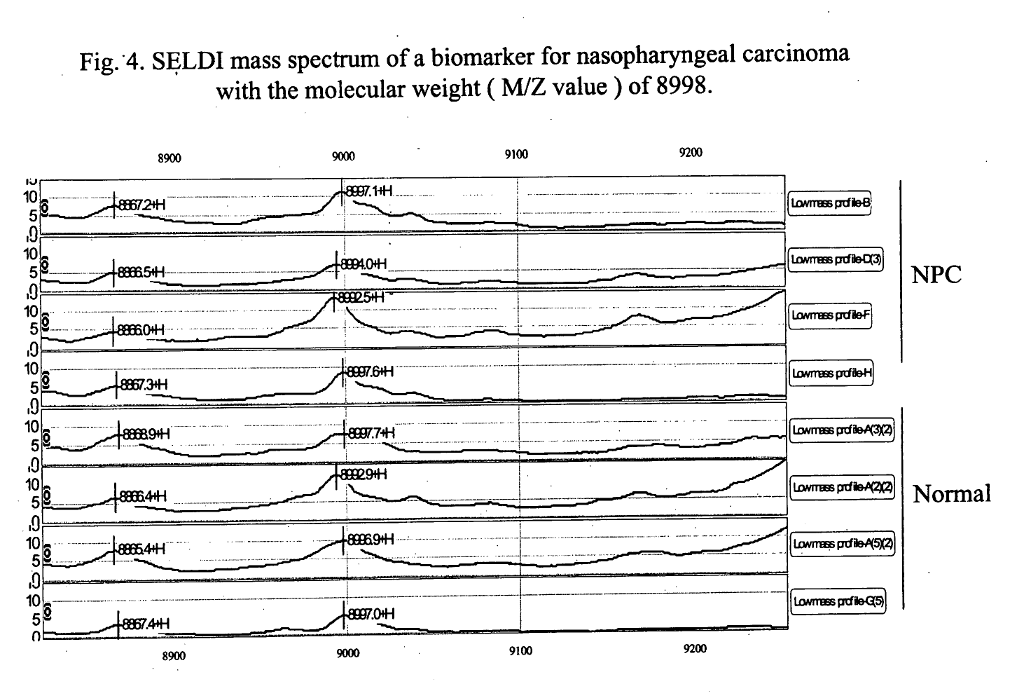

FIG. 5 is a representative SELDI-TOF-MS spectrum which characterizes a biomarker for nasopharyngeal carcinoma with the molecular weight (M/Z value) of 5391.

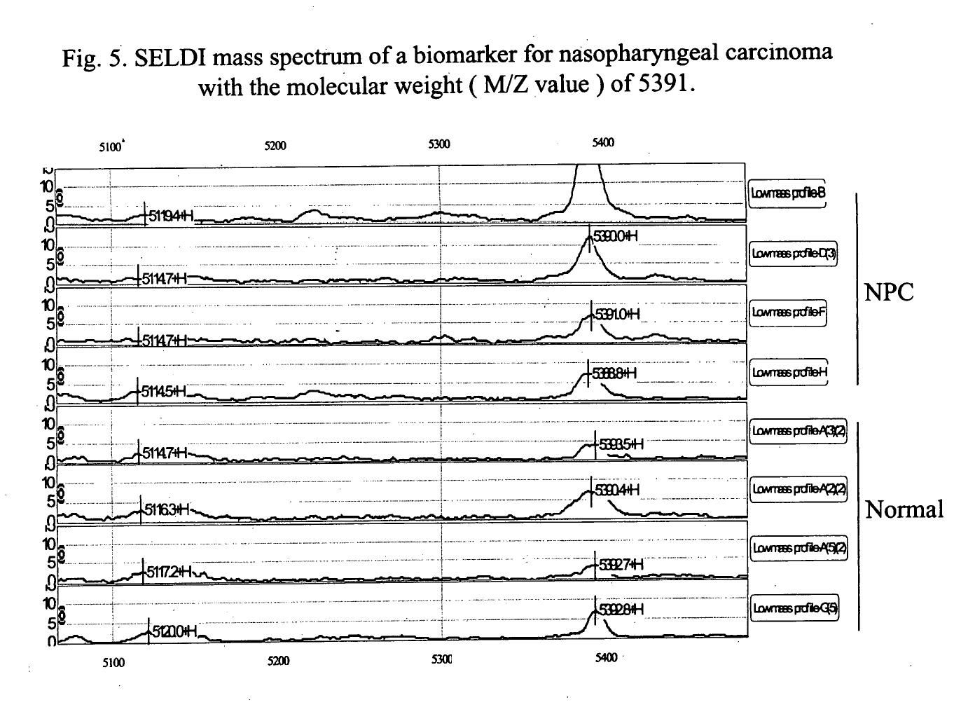

FIG. 6 is the diagram of decision tree analysis. The descendant nodes and terminal nodes within each box indicate the flow chart of diagnostic decision. Class 1 and Class 0 represent normal and nasopharyngeal carcinoma respectively. The M number under the Class represents the M/Z value of each biomarker.

### DETAILED DESCRIPTION

Since nasopharyngeal carcinoma usually contributes proteins or peptides which are related to the development and progress of nasopharyngeal carcinoma, fluid collection such as serum sample from a patient holds great diagnostic promise for the identification of cancer biomarkers.

Early detection of nasopharyngeal carcinoma can be accomplished by the analysis of serum samples using SELDI-TOF-MS. Thus, in particular embodiments the present invention uses serum sample to detect the existence and progression of nasopharyngeal carcinoma in a patient actually having the disease; to analyze proteins and peptides in the serum sample by SELDI-TOF-MS; to provide protein and peptide spectrum to set up a training algorithm; and to establish a particular protein expression profile for nasopharyngeal carcinoma.

Serum samples were collected from individuals using silica activator test tubes from BD Bioscience Co. which does not contain anti-coagulation reagents. 3-5 ml of whole blood sample was set at 4 C for 2 hours which allows a natural blood coagulation within the tube. Extra care must be taken not to disturb the tube which might cause red blood cell lysis and the release of hemoglobin, which might subsequently interfere with the protein signals on SELDI chips. Samples were then centrifuged at 1000 g for 5 min. And the serum supernatant was then transferred carefully into aliquot tubes which can be stored at 80 C for later experiment.

Following the sample preparatory steps illustrated above, various protein chip arrays were used, including but not limited to, WCX2, IMAC-3-Ni, IMAC-3-Cu, SAX2, H4, H50, PS10/PS20 and NP20, purchased from Ciphergen Biosystems (Palo Alto, Calif.). Corresponding methods for use of these protein chip arrays were available upon purchase of the protein chips. Serum samples from individuals were analyzed using SELDI-TOF MS technology, the Ciphergen Biosystem ProteinChip Reader PBS-2C.

Serum samples were taken out from 80 C refrigerator and thawed on ice. Dilute 3 ul of serum sample with 6 ul of U9 buffer (9 M urea, 2% CHAPS, 50 mM Tris-HCl, pH 9.0) and incubated on ice for 30 minutes. Tap the tubes every 5 minutes in between when incubate on ice or shake gently and continually in a cold room.

Dilute each sample into 108 ul of binding buffer (50 mM NaAc, pH 4.0) to make up a total dilution of 39×.

Assemble a chip array into an 8-well bioprocessor. Load each well with 200 ul of binding buffer, gently shake for 5 minutes. Dump the buffer and repeat the equilibration one more time.

Dump binding buffer off the chip array. Load 100 ul of diluted serum samples into each corresponding well, incubate for 1 hour with gentle shaking at room temperature.

Dump samples, load 200 ul of same binding buffer into each well, wash for 5 minutes with shaking.

Repeat the above washing step.

Dump the washing buffer, use 200 ul/well HPLC water to quick rinse the wells and dump the water.

Take out the chip arrays immediately from bioprocessor, shake off any leftover water and allow air dry.

The chip surfaces (spots) were now treated with an energy-absorbing molecule that helps in the ionization of the proteins adhering to the spots for analysis by Mass Spectrometry. The energy-absorbing molecule in this case was SPA (sinapinic acid) and a saturated solution prepared in 50% acetonitrile and 0.05% TFA. Load 0.5 ul of saturated SPA and air day.

Repeat the above SPA loading step. The solution was allowed to air dry and the chip was analyzed immediately using Ciphergen ProteinChip Reader PBS-2C.

In the present embodiment, protein chip arrays WCX2, IAMC3, SAX2, H4, H50, more preferred WCX2, were used for the establishment of the present biomarkers for nasopharyngeal carcinoma. Using ProteinChip Reader PBS-2C, the mass spectra of proteins were generated by a laser intensity of 150-190 and sensitivity of 8-10, depending on the sample variations and experimental conditions. It is a common practice to collect more than one data spectra with different reading conditions. For data acquisition of low molecular weight proteins, the detection size range was between 2 and 40 kDa.

In the present embodiment, a panel of biomarkers specific for the diagnosis of nasopharyngeal carcinoma is established. The optimum discriminatory pattern for nasopharyngeal carcinoma was defined by the amplitudes at the key M/Z (mass-to-charge ration) values. The nasopharyngeal carcinoma specific biomarkers were found as M/Z values of28383+/−142, 23650+/−118, 16128+/−81, 15741+/−79, 15310+/−77, 13930+/−70, 11836+/−59, 8998+/−45, 5856+/−29, 5690+/−28, 5531+/−28, 5391+/−27, 4172+/−24, 3208+/−16, 2833+/−14 on protein chip array of WCX2.

The biomarkers of the present invention can be used as the direct basis for diagnosis, prognosis, or treatment monitoring of a patient with nasopharyngeal carcinoma. This particular pattern of biomarkers allows a directly application in clinical diagnosis. By characterizing either the presence or absence of the said pattern of biomarkers, a diagnostician will be able to recognize nasopharyngeal carcinoma.

In certain embodiment, once cancer biomarkers are identified, assays may be employed to determine whether that these markers are present or absent in a particular sample for diagnostic, prognostic, or therapeutic purposes in a cancer patient or a patient suspected of having cancer. Assays to identify a particular protein are well-known to those of ordinary skill in the art. Such assays may involve identifying a nucleic acid encoding the marker or using an antibody that specifically recognizes the marker. Thus, the present invention concerns proteinaceous compositions that are antibodies for use in protein assays to detect the presence of a nasopharyngeal carcinoma marker.

Proteinaceous compositions may be made by techniques known to those of ordinary skill in the art, including the expression of proteins or peptides through standard molecular biological techniques, either from natural sources, or by chemical synthesis. The nucleotide, protein and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be known to those of ordinary skill in the art. Protein identification of biomarkers.

Potential biomarkers were semi-purified using micro spin columns. To prepare micro spin columns, 100 μl of QAE Sephadex A-50 (or other Sephadex products, Amersham Biosciences) pre-equilibrated with binding buffer were packed in Micro Bio-Spin columns (BIO-RAD, Hercules, Calif.).

For each sample, 20 ul of the original liver lysate were diluted into 200 μl of binding buffer and loaded on a micro spin column. The binding was carried out for 15 min in a cold room with gentle shaking. Unbound proteins flowed through columns were further washed with the same binding buffer for three times to minimize contamination.

The bound proteins were eluted with 200 μl of the same buffer at different pH. For analysis, 1 μl of each fraction was loaded directly on a normal phase (NP1) array (Ciphergen) spot and air-dried.

After addition of 0.5 μl SPA, the array was read in ProteinChip® reader. Fractions containing potential biomarkers were further separated by 4˜20% SDS-PAGE gels and corresponding bands were excised for in-gel tryptic digestion according to a protocol suggested by Ciphergen.

The resultant tryptic peptides were resolved by ProteinChip Reader and analyzed by the ProteinChip Software. The protein ID was obtained by searching NCBI database using ProFound search engine. The protein identification results were confirmed by LC-MS protocols.

Usually, if it is a known protein, the protein identification can be confirmed by LC-MS-MS (Liquide Chromotography Linker Mass Spectrometry Linked Mass Spectrometry) using the same gel slice obtained from the above protocols. Antibody can be generated using the known protein. Antibody capture assay with Ciphergen's preactived surface can be performed. The captured peak can be compared with the original biomarker peak to confirm that the two are the same protein.

If NCBI database search shows that the biomarker is an unknown protein, partial sequence can be obtained by proteases digestion and LC-MS-MS. The amino acid sequence can be converted to cDNA sequence. Corresponding cDNA clone will be obtained by cDNA library screening. Positive clone will be sequenced and amino acid sequence is confirmed by comparison. Antibody can be generated against part of the amino acid sequence and used in antibody capture assay. The captured peak is compared with the original biomarker peak to confirm that the two were the same protein.

Diagnostics in Identifying Markers.

The present invention further contemplates a method for identifying a biomarker or a panel of biomarkers for nasopharyngeal carcinoma comprising: a) collecting one set of blood samples, particularly serum samples, from confirmed nasopharyngeal carcinoma patients; b) collecting another set of serum samples from noncancerous patients or normal people; c) conducting SELDI-TOF-MS analysis and compare data between the two sets of serums; and, d) comparing the proteins and peptides profiles of serum from the first and second set of samples, wherein a serious of differences in the profiles allows the identification of nasopharyngeal carcinoma biomarkers.

In accordance with various stated objectives of the invention, the skilled artisan, in possession of the specific disease specific marker as instantly disclosed, would readily carry out known techniques in order to raise purified biochemical materials, e.g. monoclonal and/or polyclonal antibodies, which are useful in the production of methods and devices useful as point-of-care rapid assay diagnostic or risk assessment devices as are known in the art.

In preferred embodiments, the invention comprises a direct SELDI-TOF-MS analysis assay for nasopharyngeal carcinoma specific markers from serum sample. In some embodiments the invention may employ an antibody that binds immunologically to a nasopharyngeal carcinoma marker protein or peptide of the invention. In still further embodiments, the invention may further comprise subjecting proteins or peptides of the sample to ELISA.

Prognostics.

These nasopharyngeal carcinoma biomarkers of the invention can be present in serum sample of nasopharyngeal carcinoma patient by comparing biomarker profiles of a patient with a profile of a normal individual. As such, the pattern of the nasopharyngeal carcinoma markers of the invention is useful as markers in determining whether that patient's cancer will progress and, therefore, will allow a proper determination of the need for additional therapy to be made. Increasing the number of patients diagnosed in early stage of nasopharyngeal carcinoma by this invention have a direct effect on the mortality and economics of the cancer without the need to change surgical or chemotherapeutic approaches.

The presence/absence or expression level changes of the nasopharyngeal carcinoma biomarkers of the invention, will also be useful in monitoring the effectiveness of a treatment regimen. In any event, the methods of the present invention will assist physicians in diagnosing cancer and in determining optimal treatment approaches for patients of varying malignancy.

It is noted that in clinical applications, serum samples are used to screen for the presence of the biomarkers of cancer identified herein. Samples may also consist of needle biopsy cores, surgical resection samples, lymph node tissue, plasma or any other body fluids.

In other embodiments, serum samples containing nasopharyngeal carcinoma biomarker proteins would be collected from a patient and subjected to an immunoassay as described herein. Immunoassays of tissue sections are also possible.

Another embodiment of the present invention involves application of RT-PCR techniques to detect circulating cancer cells in blood (i.e., those that have already metastasized), using selected probes and primers. The presence of the nasopharyngeal carcinoma biomarker DNA in serum or lymph fluid samples is indicative of a patient with metastatic cancer, i.e., indicative of a poor prognosis.

In certain embodiment, the levels of cancer biomarkers of this invention would be compared with statistically valid groups of metastatic, non-metastatic malignant, benign or normal tissue samples; and/or with earlier marker levels in the same patient. The diagnosis and prognosis of the individual patient would be determined by comparison with such groups.

Where the presence of a particular cancer biomarker correlates with cancer progression, then the clinical detection of such a marker, or an increase in the levels of such a biomarker, in comparison to the levels in a corresponding biological sample from a normal subject or a patient of unrelated disease, is indicative of a patient with advancing cancer.

Likewise, where the absence of a cancer marker correlates with cancer progression, then the failure to clinically detect such a marker, or a decrease in the levels of such a marker, in comparison to the levels in a corresponding biological sample from a normal or a patient of unrelated disease, would also be indicative of a patient with advancing cancer.

The cancer screening method of the present invention may be readily combined with other methods in order to provide an even more reliable indication of diagnosis and prognosis. Various biomarkers of cancer have been proposed to be correlated with metastasis and malignancy.

As seen in any other diagnostic assays, detection of a panel of biomarkers for the diagnosis of nasopharyngeal carcinoma in the present invention also exhibits certain drawbacks, associated with false positives and false negatives. A false positive result occurs when an individual without malignant cancer exhibits the presence of a “cancer biomarker”.

A false negative result occurs when an individual actually has the cancer, but the test fails to show the presence of the specific biomarker pattern. For example, it is commonly seen in clinics that certain patients confirmed of nasopharyngeal carcinoma did not show any change in MRI or CT scan.

Preferred cancer biomarkers are those that are present in serum sample of nasopharyngeal carcinoma patients, and either missing or else expressed at significantly lower levels in serum of patient with benign tumors and normal individuals. As any single marker would typically be present only in some proportion of malignant cancers, it is desirable to have a number of such biomarkers for cancer.

The present invention addresses the need for nasopharyngeal carcinoma diagnosis by identifying nasopharyngeal carcinoma biomarkers that are expressed at higher levels in malignant carcinoma than in normal ones. In preferred embodiments, this invention provides nasopharyngeal carcinoma biomarkers that are indicative of cancer progression and metastatic potentials. This represents a significant advance. However, combination of the present techniques with one or more other diagnostic or prognostic techniques or markers is certainly desirable. Molecular Biological Detection Kits for Nasopharyngeal carcinoma.

Another objective of the present invention is to provide reagents for use in diagnostic assays for the detection of the particularly isolated nasopharyngeal carcinoma biomarker sequences. Any suitable direct or indirect assay method may be used to determine the level of each of the specific markers measured according to the invention. The assays may be competitive assays and sandwich assays, and the label may be selected from the group of well-known labels such as radioimmunoassay, fluorescent or chemiluminescence immunoassay, or immunoPCR technology.

The specific cancer biomarkers disclosed by the present invention are released into the blood circulation and detected in the blood or blood product, such as plasma and serum, and preparations any other body fluids, e.g. thoracic fluid, cerebral spinal fluid, saliva, urine, lymph, and the like. The presence of each marker is determined using antibodies specific for each of the markers and detecting specific binding of each antibody to its respective marker.

Kits comprising antibodies, such as antibodies to the nasopharyngeal carcinoma biomarkers, preferably their epitopes of the invention. Monoclonal antibodies are readily prepared and will often be preferred. Where cancer marker proteins or peptides are provided, it is generally preferred that they be highly purified.

Specific monoclonal antibodies for the biomarkers disclosed by the present invention may be produced, for example, by the polyethylene glycol (PEG) mediated cell fusion method, in a method well-known in the art.

Purified monoclonal antibody is utilized for immunochemical assays as molecular biological detection kits mentioned in this invention.

Polyclonal antibody production and purification utilizing one or more animal hosts by a manhood of well known in the art can be utilized in a similar manner.

For immunoassays, sandwich or double antibody assay of widely used for its simplicity of detection and quantitative nature is the preferred assay in the present invention. For example, in a typical sandwich assay, the primary unlabeled antibody is immobilized on a microtiter plate, and the tested sample is added to the plate. After the formation of an antibody-antigen complex upon incuation, a secondary antibody labeled with a reporter molecule capable of producing a detectable signal is added and allowed the binding to the antigen at a different site, resulting with a formation of a complex of unlabeled antibody-antigen-labeled antibody. The presence of the antigen is determined by observation of a signal appearance.

The immunodetection reagents of the kit may take any one of a variety of forms, including those detectable labels that are associated with, or linked to, the given antibody or antigen itself. Detectable labels that are associated with or attached to a secondary binding ligand are also contemplated. Exemplary secondary ligands are those secondary antibodies that have binding affinity for the first antibody or antigen.

In further embodiments, the invention provides an opportunity of the development of immunological kits for detection of cancer cells in biological samples such as biopsy of nasopharyngeal tissue. Such kits will generally comprise one or more antibodies that have immunospecificity for proteins or peptides biomarkers of nasopharyngeal carcinoma identified in the present invention.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Patients

Patients of nasopharyngeal carcinoma were chosen based on their confirmed pathological diagnosis by nasopharyngeal biopsy. None of the patients in the disease group has previously undergone through any radiotherapy, chemotherapy or surgery procedures. Normal individuals were eligible to participate if they have no evidence of any nasopharyngeal disease and in the corresponding sex and age group as those of patient group.

Serum sample collectionSerum samples were collected from individuals using silica activator test tubes from BD Bioscience Co., which does not contain anti-coagulation reagents. 3-5 ml of whole blood sample was set at 4 C for 2 hours which allows a natural blood coagulation within the tube. Extra care must be taken not to disturb the tube which might cause red blood cell lysis and the release of hemoglobin, which might subsequently interfere with the protein signals on SELDI chips. Samples were then centrifuged at 1000 g for 5 min. And the serum supernatant was then transferred carefully into aliquot tubes which can be stored at 80 C for later experiment.

### Example 2

ProteinChip SELDI-TOF-MS Analysis

By SELDI-TOF-MS proteomic technology (Ciphergen Biosystems), serum from pharyngeal carcinoma patients (n=35) and healthy individuals (n=40) were compared under the same testing parameters. Following the serum sample preparatory steps illustrated above, various protein chip arrays, including WCX2, IMAC-3-Ni, IMAC-3-Cu, SAX2, H4 and H50 were tried. Protein Chip WCX2 was chosen based on its performance on the identification of biomarkers specific for nasopharyngeal carcinoma.

Serum samples were taken out from 80 C refrigerator and thawed on ice. Dilute 3 ul of serum sample with 6 ul of U9 buffer (9 M urea, 2% CHAPS, 50 mM Tris-HCl, pH 9.0) and incubated on ice for 30 minutes. Tap the tubes every 5 minutes in between when incubate on ice or shake gently and continually in a cold room. Dilute each sample into 108 ul of binding buffer (50 mM NaAc, pH 4.0) to make up a total dilution of 39×. Assemble a chip array into an 8-well bioprocessor. Load each well with 200 ul of binding buffer, gently shake for 5 minutes. Dump the buffer and repeat the equilibration one more time. Dump binding buffer off the chip array.

Load 100 ul of diluted serum samples into each corresponding well, incubate for 1 hour with gentle shaking at room temperature. Dump samples, load 200 ul of same binding buffer into each well, wash for 5 minutes with shaking. Repeat the above washing step. Dump the washing buffer, use 200 ul/well HPLC water to quick rinse the wells and dump the water. Take out the chip arrays immediately from bioprocessor, shake off any leftover water and allow air dry.

The chip surfaces (spots) were now treated with an energy-absorbing molecule that helps in the ionization of the proteins adhering to the spots for analysis by Mass Spectrometry. The energy-absorbing molecule in this case was SPA (sinapinic acid) and a saturated solution prepared in 50% acetonitrile and 0.05% TFA. Load 0.5 ul of saturated SPA and air day. Repeat the above SPA loading step. The solution was allowed to air dry and the chip was analyzed immediately using Ciphergen ProteinChip Reader PBS-2C.

### Example 3

Identification of differentially expressed pharyngeal carcinoma biomarkersProtein chip array WCX2 was used for the establishment of the biomarker panel for nasopharyngeal carcinoma. Using ProteinChip Reader PBS-2C, the mass spectra of proteins were generated by a laser intensity of 150-190 and sensitivity of 8-10, depending on the sample variations and experimental conditions. It is a common practice to collect more than one data spectra with different reading conditions. For data acquisition of low molecular weight proteins, the detection size range was between 2 and 40 kDa.

A panel of biomarkers specific for the diagnosis of nasopharyngeal carcinoma is established. The optimum discriminatory pattern for nasopharyngeal carcinoma was defined by the amplitudes at the key M/Z (mass-to-charge ration) values. The nasopharyngeal carcinoma specific biomarkers were found as M/Z values of 28383±142, 23650+/−118, 16128+/−81, 15741+/−79, 15310+/−77, 13930+/−70, 11836+/−59, 8998+/−45, 5856+/−29, 5690+/−28, 5531+/−28, 5391+/−27, 4172+/−24, 3208+/−16, 2833±14 on protein chip array of WCX2.

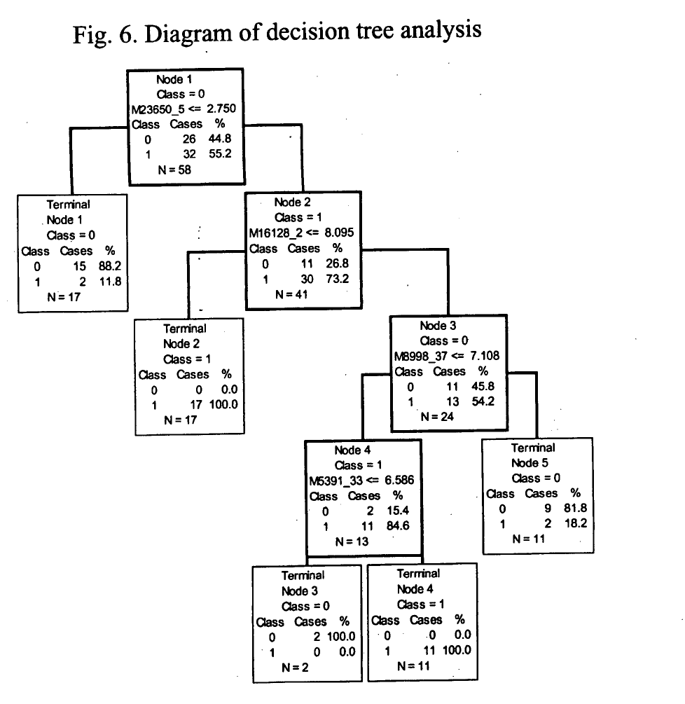

The diagnosis of nasopharyngeal carcinoma can be accomplished by the following three setups (shown in FIG. 6): a) When the intensity ratio of protein or peptide of M/Z of 23650+/−118 is less or equal to 2.7, the patient can be diagnosed as having nasopharyngeal carcinoma. 2) When the intensity ratio of protein or peptide of M/Z of 23650+/−118 is higher than 2.8, and the intensity ratio of protein of M/Z of 16128+/−81 is higher than 8.1, and the intensity ratio of protein of M/Z of 8998+/−45 is higher than 7.1, then the patient can be diagnosed as having nasopharyngeal carcinoma. 3) When the intensity ratio of protein or peptide of M/Z of 23650+/−118 is higher than 2.8, and the intensity ratio of protein of M/Z of 16128+/−81 is higher than 8.1, and the intensity ratio of protein of M/Z of 8998+/−45 is higher than 7.1, and the intensity ratio of protein of M/Z of 5391+/−27 is less or equal to 6.6, then the patient can be diagnosed as having nasopharyngeal carcinoma.

Each sample generates one mass spectrum through Ciphergen's ProteinChip Reader (SELDI-TOF-MS). Each spectrum presenting as multiple peaks (usually hundreds and thousands of peaks), with each peak representative of one protein or peptide, is processed through Ciphergen's BioWizard Software 3.0. The molecular weight of each protein or peptide can be calculated and presented as M/Z value. And the height of each peak against the baseline level representing the level (concentration) of the protein or peptide within the sample tested is demonstrated as the intensity value. A single peak or combined peaks can be used to the establishment of a diagnostic model for differential diagnosis of the cancer.

A total of 26 patients with nasopharyngeal carcinoma and 32 normal individuals were used to identify the 15 biomarkers, 28383+/−142, 23650+/−118, 16128+/−81, 15741+/−79, 15310+/−77, 13930+/−70, 11836+/−59, 8998+/−45, 5856+/−29, 5690+/−28, 5531+/−28, 5391+/−27, 4172+/−24, 3208+/−16, 2833+/−14. They offer significant differentiation values to the diagnosis of nasopharyngeal carcinoma. Among these biomarkers, four biomarkers with M/Z values of 5391+/−27, 8998+/−45, 16128+/−81, or 23650+/−118 were used to establish the current model shown in FIG. 6. The sensitive (SE), specificity (SP) and positive predictive value (PPV) were calculated as shown in Table I.

To test the model established, a double blind test was conducted using 32 samples, including samples from 18 patients of nasopharyngeal carcinoma and 14 normal individuals. The test yielded a result as shown in Table II.

TABLE I
NPC patients

| | | + | − |
|---|---|---|---|
| TEST | + | 26 | 4 |
| | − | 0 | 28 |
| | | SE: 26/26 = 100% | SP: 28/32 = 87.5% |

PPV: 26/30 = 86.7%

TABLE II
NPC patients

| | | + | − |
|---|---|---|---|
| TEST | + | 14 | 4 |
| | − | 4 | 10 |
| | | SE: 14/18 = 77.8% | SP: 10/14 = 71.4% |

PPV: 14/18 = 77.8%

1. A biomarker comprising a polypeptide specific to nasopharyngeal carcinoma.

2. The biomarker of claim 1 wherein the polypeptide having a M/A value selected from the group consisting of 28383+/−142, 23650+/−18, 16128+/−81, 15741+/−79, 15310+/−77, 13930+/−70, 11836+/−59, 8998+/−45, 5856+/−29, 5690±28, 5531+/−28, 5391+/−27, 4172+/−24, 3208+/−16, 2833+/−14, and a combination thereof:

3. The biomarker of claim 1 wherein the polypeptide having a M/A value selected from the group consisting of 23650+/−118, 16128+/−81, 8998+/−45, 5391+/−27, and a combination thereof.

4. The biomarker of claim 1 wherein the polypeptide having a M/A value selected from the group consisting of 23650, 16128, 8998, 5391, and a combination thereof.

5. A method of identifying at least one biomarker specific to nasopharyngeal carcinoma comprising the step of using a biomarker protein panel to differentiate a first serum from a nasopharyngeal carcinoma patient from a second serum from a normal subject through SELDI-TOF-MS analysis.

6. The method of claim 5 wherein said individual biomarker is one polypeptide of the biomarker panel, said polypeptide having a M/Z (mass-to-charge ration) value of selected from the group consisting of 28383+/−142, 23650+/−118, 16128+/−81, 15741+/−79, 15310+/−77, 13930+/−70, 11836+/−59, 8998+/−45, 5856+/−29, 5690+/−28, 5531+/−28, 5391+/−27, 4172+/−24, 3208+/−16, 2833+/−14, and a combination thereof on protein chip array of WCX2.

7. The method of claim 5 wherein said individual biomarker is one polypeptide of the biomarker panel, said polypeptide having a M/Z (mass-to-charge ration) value of selected from the group consisting of 23650, 16128, 8998, 5391, and a combination thereof on protein chip array of WCX2.

8. The method of claim 7 wherein a difference between the nasopharyngeal carcinoma patient and the normal subject with respect to the biomarker is measured through an intensity ratio.

9. A method for identifying a biomarker a plurality of biomarkers specific for nasopharyngeal carcinoma comprising the steps of: a) collecting a first set of blood samples, from confirmed nasopharyngeal carcinoma patients; b) collecting a second set of serum samples from noncancerous subjects; c) conducting SELDI-TOF-MS analysis for the first and second sets of serum samples; d) compare the data collected between the two serum sample sets; wherein differences in the profiles are indicative of the identification of biomarkers specific for nasopharyngeal carcinoma.

10. The method of claim 9 wherein a difference between the nasopharyngeal carcinoma patient and the normal subject with respect to the biomarker is determined by an intensity ratio.

11. A method for identifying or diagnosing nasopharyngeal carcinoma in a subject comprising the steps of 1) collecting a blood sample from a subject suspected of having nasopharyngeal carcinoma, 2) conducting SELDI-TOF-MS analysis for the blood sample and a standard blood sample, 3) comparing the data collected between the two samples; wherein a difference between the blood sample and the standard sample in at least one biomarker specific for nasopharyngeal carcinoma is indicative of the propensity for the subject having nasopharyngeal carcinoma.

12. The method of claim 11 wherein the difference with respect to the biomarker is determined by an intensity ratio.

13. The method of claim 12 wherein the intensity ratio for a biomarker having a M/Z value of 23650 is less than or equal to 2.7.

14. The method of claim 12 wherein a first intensity ratio for a biomarker having aM/Z value of 23650 is higher than 2.8, a second intensity ratio for a biomarker having a M/Z value of 16128 is higher than 8.1, and a third intensity ratio for a biomarker having a M/Z value of 8998+/−45 is higher than 7.1.

15. The method of claim 14 wherein a fourth intensity ratio for a biomarker having a M/Z value of 5391 is less or equal to 6.6.

16. A method for identifying or determining regression, progression or onset of nasopharyngeal carcinoma comprising the steps of collecting a blood sample from a subject having or suspected of having nasopharyngeal carcinoma, conducting SELDI-TOF-MS analysis for the blood sample and a standard blood sample, comparing the data collected between the two samples; wherein a difference between the blood sample and the standard sample in at least one biomarker specific for nasopharyngeal carcinoma is indicative of regression, progression or onset of nasopharyngeal carcinoma.

17. A method for evaluating the effect of a drug candidate for nasopharyngeal carcinoma comprising collecting a blood sample from a subject having nasopharyngeal carcinoma and being administered with the drug candidate, conducting SELDI-TOF-MS analysis for the blood sample and a standard blood sample, comparing the data collected between the two samples; wherein the reducing, sustaining or increasing of a difference between the blood sample and the standard sample in at least one biomarker specific for nasopharyngeal carcinoma is indicative of the effect of the drug candidate.

18. A method for post-operatively monitoring cancer prognosis and occurrence comprises: using a serum sample from said subject to develop a post-operative biomarker panel; comparing said post-operative biomarker panel with a pre-operative biomarker reference panel for said subject; and determining the absence or still presence of malignancy by monitoring at least one constituent of said biomarker panels.

19. A method of using the intensity value of a biomarker to diagnose pharyngeal carcinoma comprising: using serum sample from an individual to provide a method of cancer diagnosis; comparing intensity value of said individual protein biomarker with a reference protein intensity value; and determining the alteration of intensity value of said individual protein biomarker over said reference protein to diagnose said subject.
